(19) United States
(12) Patent Application Publication
(10) Pub. No.: US 2013/0036554 A1
(43) Pub. Date: Feb. 14, 2013

(54) Pregnancy Sleep-Aid

(71) Applicant: Elisa Steinmetz, Eau Claire, WI (US)

(72) Inventor: Elisa Steinmetz, Eau Claire, WI (US)

(21) Appl. No.: 13/088,371

(22) Filed: Aug. 9, 2011

(51) Int. Cl. A47C 20/00; A61B 19/00

(52) U.S. Cl. 5/631; 128/898

(57) ABSTRACT

The invention includes a belt that a pregnant woman wears while sleeping to support her abdominal area when she is lying on her side. There are two ribbon strips that wrap around the abdominal area and fasten in the front. Two memory foam pillows are attached to the belt to support either side of the woman's abdominal area. When lying down, the skinniest park of the pillow will be at the woman's back and the wider part is under her abdominal area to provide extra support to the baby. There are two extension strips included to ensure that the belt will fit around any size abdominal area.

### BACKGROUND

1. Field of the Invention

The present invention relates generally to sleep aids, and more specifically to sleep aids that provide support for a women during pregnancy.

2. Background Information

While a standard pillow is often sufficient to accommodate the sleep comfort of most individuals, sometimes additional devices are used or needed. There are various sleep aid devices on the market. However, there is always room for improvement.

### SUMMARY OF THE INVENTION

The sleep aid as described in this specification is a device designed to help individuals who need to comfortably accommodate a larger abdominal area, especially when sleeping. The device is especially helpful for pregnant women. The pregnancy sleep aid device needs to be adjustable to fit various sizes of women and should be easily adjustable for different sizes or growth of the abdominal area. In accordance with an aspect of the invention, the device is directed toward a sleep aid that is adjustable to individual needs. Generally, the inventive device includes a strap or straps with hook-and-loop, i.e., Velcro® brand strips, fixed at the ends of a strap that connects to pads or pillows that may easily be removed and/or affixed.

Applicant appreciates that it is often advisable or preferable for a pregnant mother to avoid laying on her back. In operation, an embodiment of the invention may include sliding a strap through an opening of a foam pad. Once the foam pad is in place a second foam pad may slide onto the strap through an opening of the second foam pad. The two movable foam pads can then be positioned into place to provide support for the abdomen area of the mother while the mother is lying on her side.

Further, the device and method may take on any configuration that is capable of being retrofitted onto an abdominal area so as to connect the strap or straps with the foam pads in a manner that provides comfort for the pregnant woman while lying on her side.

An object of the invention is to provide a device that allows for comfortable rest for persons having a large abdomen.

A further object of the invention is to provide a device that allows for comfortable rest for a pregnant women while lying on her side.

An object of the invention is to provide a device that is simply and easily fitted onto the abdominal area and simply and easily adjusted to fit the size of the abdominal area.

A further object of the invention is to provide a device that is comfortable for a user to wear.

The above summary of the present invention is not intended to describe each illustrated embodiment, aspect, or every implementation of the present invention. The figures and detailed description that follow more particularly exemplify these and other embodiments and further aspects of the invention.

### BRIEF DESCRIPTION OF THE DRAWINGS

The invention may be more completely understood in consideration of the following description of various embodiments of the invention in connection with the accompanying drawings, in which:

FIG. 1 is a perspective view of one embodiment of the invention.

FIG. 2 is a perspective view of the device f FIG. 1 in a connected mode.

FIG. 3 is a perspective view of features of the invention.

FIG. 4 is a perspective view of a feature of the invention.

FIG. 5 is a reverse perspective view of the feature of FIG. 4.

While the invention is amenable to various modifications and alternative forms, specifics thereof have been shown by way of example in the drawings and will be described in detail. It should be understood, however, that the intention is not necessarily to limit the invention of the particular embodiments described.

### DETAILED DESCRIPTION OF THE INVENTION

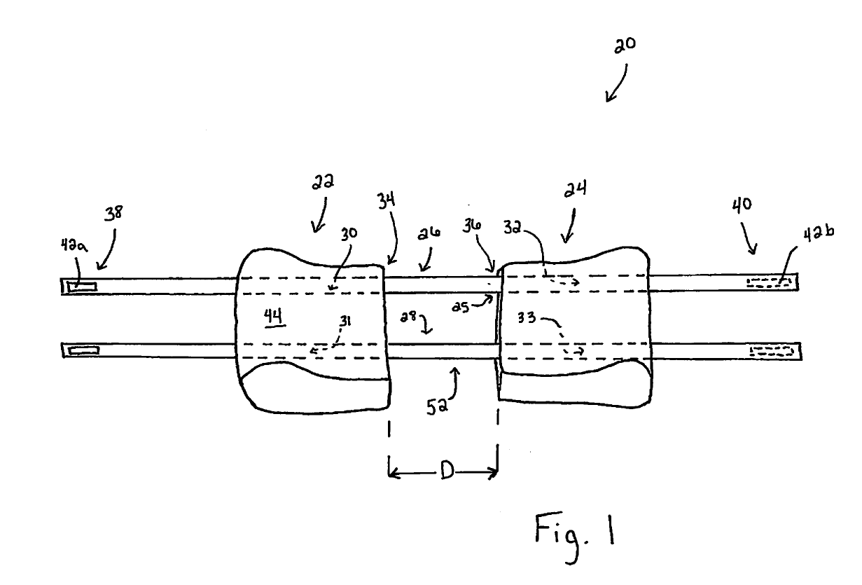

Referring now to FIGS. 1-5, the invention is directed to a sleep aid device 20 and methods of using sleep aid device 20.

Generally, device 20 comprises a first pad 22 and a second pad 24. Pad 22 and pad 24 may be identical or may be of different shapes or made of different materials. A first strap 26 connects first pad 22 to second pad 24. Preferably a second strap 28 is also provided to connect first pad 22 to second pad 24. Preferably pads 22, 24 are of a “memory foam” variety in that they retain their shape after being compressed or stretched. A cover may be placed over pads 22, 24. Different types of materials may be used for pads 22, 24. Preferably pads 22, 24 have a curved portion 50 which allows for different comfort orientations to be achieved by a user. Curved portion 50 may have a thickness greater than a narrower opposite end.

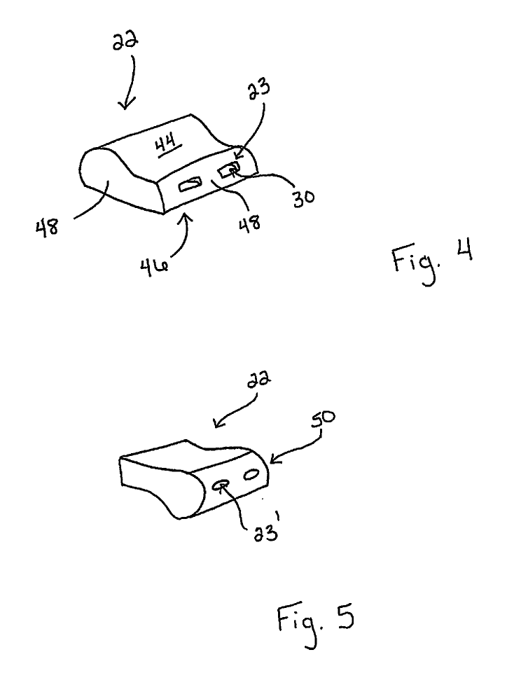

As shown in FIG. 1, first pad 22 is positioned a distance D from second pad 24. In one aspect, first strap 26 slides through a first pad channel 30. Strap 26 enters first pad opening 23 and extends through channel 30 where it exits second opening 23′ (See FIG. 4, FIG. 5). Strap 26 may slide through and be adjusted along channel 30. Preferably, first strap 26 also slides through second pad channel 32 such that first pad 22 is positionable a distance D from second pad 24, creating a middle portion 52. Strap 26 may also slide through and be adjusted along channel 32. Preferably device 20 includes second strap 28 which slides through second first pad channel 31 and through second second pad channel 33, similarly as described with respect to strap 26. It may be appreciated that various sizes of straps and channels may be used to accommodate differences in preference. Additional straps and/or channels may also be used in conjunction with device 20. In one aspect strap 26 may be a polyester ribbon that is flexible. Straps 26, 28 may be of any or a variety of colors and textures, and it may be of a variety of elasticity.

A channel 30, 31, 32, 33 may be sized to accommodate a variety of different styles of straps 26, 28. A channel may also be present as a sleeve positioned upon pad 22, 24, such as created in a space between a cover and the pad 22. Preferably, however, channel 30 runs through the body of a pad 22.

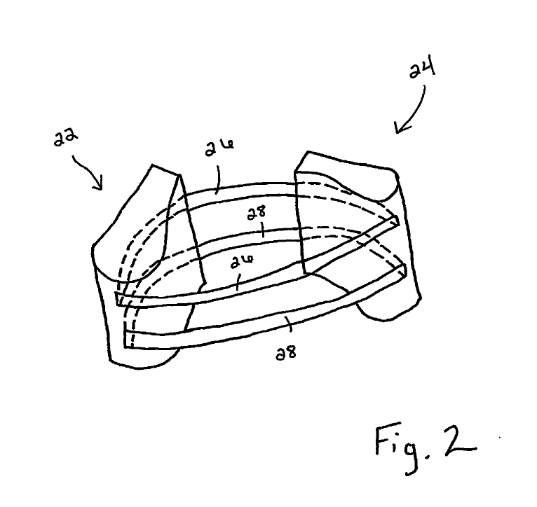

The first strap 26 and second strap 28 as shown in FIGS. 1 and 2 have an unstressed width of about 1 inch and an unstressed length of about 50 inches. A hook-and-loop fastener component 42 is fixed at the ends of the extension strip 54. The hook-and-loop fastener component 42 may have multiple components 42a and 42b. In one aspect, first component 42a may comprise the hook component, preferrably located at first end portion 38. Second component 42b may comprise the loop component, preferrably located second end portion 40. First end portion 38 may connect to second end portion 40. Thus the first strap 26 and second strap 28 can be positioned to encircle the abdominal area of a women with the top surface 44 of pad 22 oriented toward the abdominal area, and the bottom surface 46 of pad 22 oriented towards the outside or away from the wearer as seen in FIG. 2. It may be appreciated that pad 22 may be positioned a first distance D from second pad 24. It may also be appreciated that first strap 26 may slide through first pad such that first pad 22 is positionable a second distance from second pad 24. Second distance may be of a length that is greater or less that first distance D. In this way pad 22, 24 may be adjusted to accommodate the comfort of each individual user, who may also reposition pads 22, 24 as desired. Pads 22, 24 are independently moveable along strap 26, 28. It may be appreciated that channels 30, 31 may be parallel or may have some other orientation.

First pad 22 shown in FIGS. 4 and 5 is preferably made out of a generally curved pad having a curved portion 50. First pad 22 may comprise of first pad opening 23 for receiving first strap 26 with a second opening 23′ for receiving second strap 28. Preferably first pad opening 23 is positioned at a perimeter edge 48 of pad 22. First pad opening defines a first end 34 of first pad channel that extends through the first pad 22. Second pad opening 25 defines a first end 36 of second pad channel that extends through second pad 24.

A pad 22, 24 may be of many sizes. In one aspect pad 22 may have a width of about 10 inches (running from a left perimeter edge to a right perimeter edge) and a length running from a front perimeter edge 48 to a rear curved portion of about 8 to 9 inches (preferably about 9 inches, and preferably about 8 inches as measured from the thickest portion of pad 22 to front perimeter edge 48). A thickness of pad 22 at curved portion may be about 4-5 inches (preferably about 4¾ inches, and a narrower thickness at an opposite end of about 3 inches). The particular dimensions of pads 22, 24 may vary as desired.

A third pad may also be used. Third pad may be positioned at middle portion 52. Third pad may be a pad or pillow that is similar to pads 22, 24. Third pad may be used to assure the wearer does not continue to lie on her back; instead the wearer will be coaxed to lay on her side. Pads 22, 24 are preferably positioned and configured to support the belly area of the person. In the case of a pregnant mother, pads 22, 24 assist in support of the baby.

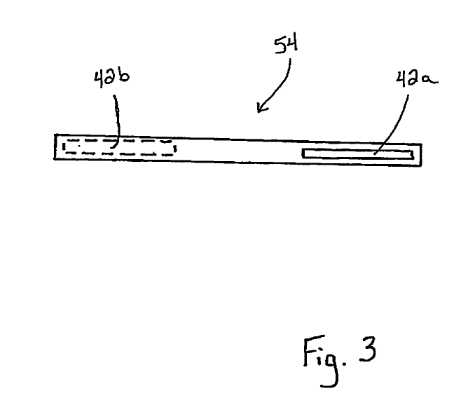

An extension strip 54 shown in FIG. 3 has an unstressed width of about 1 inch and an unstressed length of about 24 inches. Strip 54 may be of any desired length, and preferably is about two feet in length. A hook-and-loop fastener component 42 is fixed at the ends of the extension strip 54. The hook-and-loop fastener component 42 may have multiple components 42a and 42b. First component 42a may comprise the hook component, preferrably located at opposite end of 42b. Second component 42b may comprise the loop component, preferrably located at opposite end of 42a. Extension strip 54 may be used to increase the length of a strap 26, 28 to accommodate women having different sizes of abdomens.

One aspect of the invention includes a method of using the device 20 by affixing the device 20 to the abdomen of a person. The pads 22, 24 help the wearer obtain a comfortable sleeping position.

The terms and descriptions used herein are set forth by way of illustration only and are not meant as limitations. Those skilled in the art will recognize that many variations are possible within the spirit and scope of the invention as defined in the following claims, and their equivalents, in which all terms are to be understood in their broadest possible sense unless otherwise specifically indicated. While the particular SLEEP AID AND METHOD as herein shown and described in detail is fully capable of attaining the above-described aspects of the invention, it is to be understood that it is the presently preferred embodiment of the present invention and thus, is representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. section 112, sixth paragraph, unless the element is expressly recited using the phrase “means for.”

1. A device comprising:
a first pad having a first pad opening for receiving a strap;
a second pad having a second pad opening for receiving a strap;
a first strap extending from said first pad opening to said second pad opening to connect said first pad to said second pad.

2. The device of claim 1 where said first pad opening defines a first end of a first pad channel that extends through said first pad and where said second pad opening defines a first end of a second pad channel that extends through said second pad, said first strap extending through said first pad channel and through said second pad channel.

3. The device of claim 2 where said first strap has a first end portion and a second end portion, said first end portion connects to said second end portion.

4. The device of claim 3 where said first end portion contains a component of a hook-and-loop fastener and said second end portion contains a component of a hook-and-loop fastener.

5. The device of claim 3 further comprising a second strap extending from said first pad to said second pad.

6. The device of claim 5 where said first pad includes a second first pad channel and said second pad includes a second second pad channel, said second strap extending through said second first pad channel and said second second pad channel.

7. The device of claim 6 where said first strap slides through said first pad channel.

8. The device of claim 7 where said second strap slides through said second pad channel.

9. The device of claim 8 where said second strap has a first end portion and a second end portion, said first end portion connects to said second end portion.

10. The device of claim 1 where said first pad and said second pad are movable memory foam pillows.

11. The device of claim 10 where said pillows are independently moveable along said first strap.

12. The device of claim 2 where said first pad is positioned a first distance from said second pad, said first strap slides through said first pad channel such that said first pad is positionable a second distance from said second pad.

13. The device of claim 12 where said first strap slides through said second pad channel.

14. The device of claim 1 further comprising a third pad positioned on said first strap between said first pad and said second pad.

15. The device of claim 1 where said first pad includes a top surface and a bottom surface separated by a perimeter edge, said first pad opening positioned at said perimeter edge.

16. The device of claim 15 where said first pad includes a first pad channel extending from said first pad opening through said first pad to a second opening positioned on a generally curved portion of said first pad.

17. A method comprising affixing to the abdomen of a person the device of claim 1.

18. The method of claim 17 where said first strap has a first end portion and a second end portion, a middle portion of said first strap which lies between said first pad and said second pad is positioned against a back of a person, and connecting said first end portion to said second end portion.

19. The method of claim 18 where said device is connected to the abdomen area of a pregnant woman to support a belly of the women laying down on her side.

20. The method of claim 19 where said first strap extends across the belly of the pregnant woman.
